(19) United States
(12) Patent Application Publication
(10) Pub. No.: US 2025/0009857 A1
(43) Pub. Date: Jan. 9, 2025

(54) NUTRACEUTICAL COMPOSITION BASED ON PINEAPPLE AND PLANT EXTRACTS FOR THE TREATMENT OF IRRITABLE BOWEL SYNDROME

(71) Applicant: Autonomous University of Nuevo Leon (UANL) (Universidad Autonoma de Nueva Leon), Ciudad Universitaria (MX)

(72) Inventor: Adbel Zaid MARTINEZ BAEZ, Ciudad Universitaria (MX)

(21) Appl. No.: 18/765,199

(22) Filed: Jul. 5, 2024

(30) Foreign Application Priority Data
Jul. 6, 2023 (MX) MX/A/2023/008088

(51) Int. Cl. A61K 38/48; A61K 9/06; A61K 36/52; A61K 36/61; A61K 36/88; A61K 47/36; A61P 1/14

(52) U.S. Cl. CPC A61K 38/4873; A61K 9/06; A61K 36/52; A61K 36/61; A61K 36/88; A61K 47/36; A61P 1/14

(57) ABSTRACT

The present invention provides a nutraceutical gel based on pineapple (Ananas comosus), guava (Psidium guajava), and walnut (Juglans regia), which, when consumed, has a digestive and/or anti-inflammatory effect on patients with irritable bowel syndrome. It proposes an alternative dietary supplement for those suffering from this condition and has been evaluated through a clinical study. The natural products used possess nutraceutical characteristics that help meet the specific nutritional needs of patients with irritable bowel syndrome.

### OBJECT OF THE INVENTION

The object of the present invention is to provide a nutraceutical composition, particularly in the form of a nutraceutical gel based on pineapple (Ananas comosus) and plant extracts, specifically guava (Psidium guajava) and walnut (Juglans regia) and its use for the treatment of patients with irritable bowel syndrome. The present invention belongs to the technical field of natural products with applications in gastrointestinal health.

### BACKGROUND

Irritable bowel syndrome (IBS) is defined as a chronic affectation of abdominal pain or discomfort associated with alterations in the intestinal habit, for at least 3 days per month in the last 3 months, without organic cause that justifies it. Its etiology is unknown and it is believed to have a multifactorial cause in which different factors may coexist. Different subtypes have been identified, either predominantly diarrhea (IBS-D), constipation (IBS-C) or mixed (IBS-M), and it seriously affects the quality of life of those who suffer from it.

Digestive problems affect people of all ages, and it is estimated that between 16%-30% of the population suffers from colon or irritable bowel syndrome (IBS); it mainly affects women under 45 years of age. IBS is a functional disorder characterized by abdominal pain or discomfort associated with alterations in bowel habit and other gastrointestinal symptoms such as bloating and a feeling of abdominal swelling, incomplete evacuation, urgency, straining, and tenesmus. Many of these disorders can be controlled with changes in diet and lifestyle, so nutrition is a key element in their prevention and treatment. Studies conducted in Mexico have found that the most frequent subtype of this disorder is one in which constipation predominates (IBS-E), followed by the alternating or mixed type (IBS-M). Although several good quality studies on the epidemiology of IBS have been carried out in Mexico, none of them establish the number of new cases of the disease that arise in a given period of time, so the incidence of this functional disorder in our country is unknown to date.

The clinical symptoms individually are imprecise for establishing the diagnosis of IBS, so this pathology should be considered as a syndromic whole. The association of chronic abdominal pain and altered bowel habits are the main non-specific features of IBS, although there is a wide variety of symptoms including gastrointestinal and extraintestinal manifestations. The main gastrointestinal symptoms are: chronic abdominal pain, altered bowel habits (diarrhea or constipation), and altered bowel habits (diarrhea or constipation or alternation of these), gastroesophageal reflux, dysphagia, early satiety, intermittent dyspepsia, nausea, precordial pain of non-cardiac origin, flatulence and belching, while the extraintestinal symptoms are: major depression, anxiety, somatoform disorder, insomnia, sexual dysfunction and dyspareunia, dysmenorrhea, increased urinary frequency, urinary urgency, asthma symptoms, and primary headache. [Gupta C, Prakash D. Nutraceuticals for geriatrics. Afr J Tradit Complement Altern Med 2015; 5:5-14].

According to the WHO, approximately 80% of the world's population uses natural products for medicinal purposes. Between 1983 and 1994, the FDA approved 520 new drugs, 39% of which were of natural origin. Because in nature there is a wide variety of bioactive molecules, which are called active principles, due to the fact that these molecules, which are the product of the metabolism of plant organisms, possess pharmacological activity and are susceptible to therapeutic use. It is observed that in most cases the whole plant is more biologically active than the sum of its isolated active principles, essentially due to the interaction of different active principles of the plant and its biological components. Pharmaceutical experimentation has shown that the action of a plant cannot be explained by the action of one of its active principles. Natural health products are those that are administered orally and contain one or more active ingredients that are considered nutrients or herbal products. [Boullata, J. (2005). Natural health product interactions with medication. Nutrition in Clinical Practice, 20 (1), 33-51. https://doi.org/10.1177/01154265050200013].

Among the natural products used for medicinal purposes are functional foods and nutraceuticals, consumed as part of a balanced diet and accompanied by a healthy lifestyle, they offer the possibility of improving health and/or preventing certain diseases. The term functional food/product defines foods/products for specific health use. Among the existing definitions of functional product is that of the International Food Information Council, which defines it as “any food similar in physical appearance to conventional food, consumed as part of the daily diet, but capable of producing demonstrated metabolic or physiological effects, useful in maintaining good physical and mental health, in reducing the risk of chronic degenerative diseases, in addition to its basic nutritional functions” [Reglero, G. (2011). Novel foods, functional foods and nutraceuticals. Nutrition, health and functional foods. https://doi.org/10.1017/S0007114599000471].

Likewise, we find food supplements, which in the category of goods and services of the General Health Law are defined as a product based on herbs, vegetable extracts, traditional foods, dehydrated or concentrated fruits, with or without added vitamins or minerals, which can be presented in pharmaceutical form and whose purpose of use is to increase the total dietary intake, complement it or supply some component, according to article 215, section V, of the General Health Law. The pharmaceutical forms accepted are those that are ingested orally such as: gel, capsule, emulsion, suspension, syrup, powder, solutions and tablets, among others contemplated in the Pharmacopoeia of the United Mexican States.

### Pineapple (Ananas comosus)

The pineapple is an herbaceous plant of 1 to 1.5 meters in extension, both in height and in circumference. It is formed by a rosette of hard leaves, lanceolate and more or less thorny, organized around a stem that constitutes the axis of the plant. In its extension grows an apex at the end of which is born the fruit ending in a crown. The pineapple produces few seeds, so its reproduction, especially in industrial plantations is made from the shoots produced by the plant after fruiting. [Dawson C. (2000). PIÑA. 27/08/20, from NATIONS UNIES CNUCED Sitio web: https://unctad.org/es/PublicationsLibrary/INFOCOMM_cp09_Pineapple_es.pdf].

This plant is composed of 81 to 86% water, so its caloric value is low, leaving 13 to 19% of total solids, among which are disaccharides such as sucrose and monosaccharides such as glucose and fructose, together carbohydrates represent up to 85% of total solids and fiber from 2 to 3%. Containsglycine (32.2%), serine (32%) and aspartic acid (29.8%) at high levels, while histidine (1.3%), methionine (5.8%) and phenylalanine (8%) are present at lower levels. Minerals include iodine, potassium, magnesium, copper, manganese and calcium. The most abundant vitamins are vitamin A and C, and to a lesser extent thiamine or B1 and pyridoxine or B6. [Hossain, F., Akhtar, S., & Anwar, M. (2015). Nutritional Value and Medicinal Beneficits of Pineapple. International Journal of Nutrition and Food Sciences, 84-88].

In addition, pineapple contains Bromelain, which is an enzyme that may have antioxidant virtues and is widely used as an anti-inflammatory compound due to its protease activity. It is classified as an endopeptidase belonging to the Peptidase CIA subfamily. It may refer to a mixture of cysteine proteinases, peroxidases, acid phosphatases, glycosidases and inhibitory proteins. Found throughout the pineapple fruit and stem. It has clinical effects by oral administration in cancer, rheumatoid arthritis and osteoarthritis. [Cantú M. L., Hernández M., Medina I. G. & Martínez A. Z. (2019, diciembre 26). Aplicaciones terapéuticas y composición quimica de Ananas comosus (L.) Merr. (PIÑA). Un estudio de revisión. Planta, Año 15 No. 26, pp. 15-24].

It has also been identified that this plant contains compounds with biological antirheumatic activity. [Kargutkar, S., & Brijesh, S. (2016). Anti-rheumatic activity of Ananas comosus fruit peel extract in a complete Freund's adjuvant rat model. Pharmaceutical Biology, 2616-2622. https://www.ncbi.nlm.nih.gov/pubmed/27181794; Bhattacharya, B. (2008). Bromelain: An overview. Natural Product Radiance, 359-363. https://pdfs.semanticscholar.org/4a4d/fdle0e0f4774aaedfa0a6alaf9408f9ec755.pdf], anti-inflammatory [Rathnavelu, et al. 2016; Aiyegbusi, A., Doru, F. I., Anonobi, C. C., Noronha, C. C., & Okanlawon, A. O. (2011). Bromelain in the early phase of healing in acute crush Achilles tendon injury. Phytotherapy Research, 49-52], anticarcinogenic and antimicrobial [Rathnavelu, V., Banu, N., Sohila, S., Kanagesan, S., & Ramesh, R. (2016). Potencial role of bromelain in clinical and therapeutic applications. Biomedical Reports, 283-288. https://www.ncbi.nlm.nih.gov/pmc/articles/PMC4998156].

### Guava leaf (Psidium guajava)

Popularly known as guava, it is a small tree belonging to the myrtle family (Myrtaceae). Native to tropical areas from southern Mexico to northern South America, many other countries with tropical and subtropical climates have cultivated guava trees, allowing for worldwide production. Its growing habitat is within the tropical and subtropical regions, being highly versatile in terms of height of cultivation (0 to 1515 meters above sea level) and can be grown in any type of soil, tolerating a pH range of 4.5 to 9.4. (Antonio Yam Tzec, José, Villaseñor Perea, Carlos Alberto, Romantchik Kriuchkova, Eugenio, Soto Escobar, Martin, & Peña Peralta, Miguel Ángel. (2010). A review on the importance of guava fruit (Psidium guajava L.) and its main post-harvest characteristics. Revista Ciencias Técnicas Agropecuarias, 19 (4), 74-82. Retrieved February 11, 2022, from http://scielo.sld.cu/scielo.php?script=sci_arttext&pid=S2071-00542010000400012&Ing-es&tlng=es).

The maturity of guava influences its chemical composition as shown in Table 1.

TABLE 1
Chemical composition (%) of traditional red guava and its pulp.

| Component | Red guava | Red ripe guava | Guava pulp |
|---|---|---|---|
| Moisture | 76.2-90.90 | 77-86 | 78-86 |
| Protein | 0.6-1.60 | 0-9-10 | 0.45-1.5 |
| Fat | 0.35-0.070 | 0.1-1.0 | 0.1-0.9 |
| Carbohydrates | 2.41-14.19 | 9.5-10 | 9.0-12.0 |
| Fiber | 2.69-5.15 | 2.8-5.5 | 1.0-3.0 |
| Ash | 0.34-0.095 | 0.4-0.7 | 0.5-1.0 |

Several metabolites with good performance and some have been shown to possess useful biological activities belonging mainly to phenolics, flavonoids, carotenoids, terpenoids and triterpenes. [Gutiérrez R M, Mitchell S, Solis R V. Psidium guajava: a review of its traditional uses, phytochemistry and pharmacology. J Ethnopharmacol. 2008;117 (1): 1-27. doi: 10.1016/j.jep.2008.01.02].

There is scientific evidence that guava has bioactive compounds that have biological, antibacterial, anti-inflammatory, antidiarrheal, antidiabetic and have biological, antibacterial, anti-inflammatory, antidiarrheal, antidiabetic, and for some topical lesions. In Mexico, scientific works related to alternative uses in gastrointestinal diseases have been reported [Díaz-de-Cerio E, Verardo V, Gómez-Caravaca A M, Fernández-Gutiérrez A, Segura-Carretero A. Health Effects of Psidium guajava L. Leaves: An Overview of the Last Decade. Int J Mol Sci. 2017;18 (4): 897. Published 2017 Apr. 24. doi: 10.3390/ijms18040897; Birdi T, Krishnan G G, Kataria S, Gholkar M, Daswani P. A randomized open label efficacy clinical trial 5 of oral guava leaf decoction in patients with acute infectious diarrhoea. J Ayurveda Integr Med. 2020;11 (2): 163-172. doi: 10.1016/j.jaim.2020.04.001].

### Walnut Leaves (Juglans regia)

The walnut is the most widespread and well-known species of the Juglandaceae family. It is a vigorous tree with a solid, tall, straight trunk, which opens into a leafy, wide and harmonious crown. The leaves are compound, deciduous, of an intense green color, balsamic odor and astringent bitter taste. The female flowers are solitary or gathered in groups of 2-3 and appear on the new shoots of the year, simultaneously with the leaves. The fruit is a globose drupe. The seed, rich in oils, is divided into 4 rough and irregular lobes and ripens in April-May [Veronica Loewe M. (1991), Apuntes sobre algunas latifoliadas de maderas valiosas. Vol. 5 Num 1/165. INSTITUTO FORESTAL, Santiago de Chile https://bibliotecadigital.infor.cl/bitstream/handle/20.500.12220/10809/18507.pdf?sequence=1&is Allowed=y]

Walnut leaves contain tannic matters, ellagic acid and gallic acid and, when fresh juglone which is a naphthoquinone formed secondarily from α-hydrolone and inosite. In the epi and mesocarp there is also hydrolin. The seed contains up to 50% oil (linoleic acid predominates) and sucrose, dextrose, dextrin,starch, pentosans, the globulin yuglansin, lecithin, inositopentaphosphoric acid, etc. [Divina Aparicio (2016). Walnut tree (Juglans regia L.). 11 02 2021, from Virtual Biodiversity Website: https://www.biodiversidadvirtual.org/etno/Nogal-(Juglans-regia-L.)-img30983.html]

Walnut leaves present oleic and cationic tannins, which provide astringent properties. They also present quinonic derivatives such as juglone, justifying antiseptic and healing properties of interest to treat eczema. Ellagic tannins (10%), Naphthoquinones: juglone and hydro juglone (traces in the dry drug). Traces of essential oil with D-germacrene, flavonoid derivatives: hyperoside, juglanin, quercetin., phenyl carboxylic acids: caffeic, gallic, neo chlorogenic, ascorbic acid (1%) [Divina Aparicio. (2016). Walnut (Juglans regia L.). 11 02 2021, from Virtual Biodiversity Website: https://www.biodiversidadvirtual.org/etno/Nogal-(Juglans-regia-L.)-img30983.html Nael A & Mohammed A. (2011 December 30). Utility and importance of walnut, Juglans regia Linn: A review. African Journal of Microbiology Research, 5 (32), pp. 5796-5805. 2022 02 15, From Google Scholar Database].

The tannins present in walnut give it astringent (antidiarrheal, local hemostatic, wound healing, antisudoral) and antiseptic properties. It is also used as an eupeptic, cholagogue, mild hypoglycemic, anthelmintic and depurative. Traditional uses approved by the French Ministry of Health: internal use: symptomatic relief of venous insufficiency, hemorrhoids and mild diarrhea, topical use: head itching, dandruff, as an adjuvant in cases of pruritus, hematomas, diaper rash, insect bites, superficial burns and inflammations of the oral or pharyngeal mucosa, and other popular uses: mild diabetes, rheumatism, gout, inappetence, hypo-secretory dyspepsia; in topical use: wounds and dermal ulcerations, blepharitis, conjunctivitis, eczema, erythema, vulvovaginitis.

### Edible Gels

Although gels are strictly colloidal dispersions, they are very important in the food industry because of their characteristics. Gels result from the solidification of a sol (or colloid) (colloidal system formed by a solid dispersed phase in a liquid dispersant), and are considered as a matrix or network of interconnected macromolecules that trap and immobilize the liquid phase in their spaces. Gels exhibit varying degrees of stiffness and elasticity, and exhibit semi-solid and solid structures. The mechanism of sol-to-gel conversion is not entirely clear, so numerous theories and models have been proposed. What is important is that the resulting gel exhibits a solid or semi-solid state by immobilizing the dispersing phase of the initial sol. The relative amounts of dispersed and dispersant phase in a sol that give rise to a gel vary widely. Generally, the dispersant (liquid) phase is in higher concentration and even then, when the gel is obtained, it is immobilized and the final structure has some degree of rigidity and elasticity. For example, agar-agar gels are solid and elastic structures with a water content that can be up to 99.9% [Montejano Gaitán, J. G. (2012). Dispersion state. In S. Baudi Dergal (Ed.), Food chemistry (pp. 560-561). Nahucalpan de Juárez, Edo. de México, Mexico: Person].

Some gels can be thermoreversible, i.e., they can be melted, returning them to a sol-type dispersion, thanks to the addition or elimination of thermal energy. In general, in these gels the bonds that maintain the three-dimensional structure are of the hydrogen bridge and Vander Waals type. For example, gelatin or agar gels are examples of these, since they can pass from the solid to the liquid state by heating. When covalent bonds and hydrophobic interactions are present in the structural molecules, as in the case of proteins and some polysaccharides, the gel is thermo irreversible; for example, cooked egg white and meat sausages [Montejano Gaitán, J. G. (2012). Dispersion state. In S. Baudi Dergal (Ed.), Food chemistry (pp. 560-561). Nahucalpan de Juárez, Edo. de México, Mexico: Person].

A sol can be transformed into a gel through physical and chemical mechanisms. Thermal energy and high pressures are considered physical agents, while the chemical agents that can induce gelation are: a) ions, such as calcium for rubber gelation; b) organic and inorganic acids, for the production of restructured meat; c) enzymes, such as bacterial transglutaminase, used in surimi-based products; and d) urea, with which transparent egg white or bovine albumin gels can be produced; However, its use is rare at an industrial level. [Montejano Gaitán, J. G. (2012). Estado de dispersión. En S. Baudi Dergal (Ed.), Química de los alimentos (pp. 560-561). Nahucalpan de Juárez, Edo. De México, México: Person].

Now, most gels absorb water, however, some of them release part of their liquid phase, without taking into account the vapor pressure exerted on them. This physical phenomenon is called syneresis. It should be noted that the liquid released in syneresis is generally a dilute sol.

Syneresis in gels is influenced by several factors: a) it is affected by the pH of the system and reaches its maximum when the isoelectric point occurs; For example, in the manufacture of fruit jellies, adequate gelation and firmness largely depends on achieving a pH between 3.2 and 3.5, a lower pH will cause a very rigid structure and syneresis will occur; b) the temperature at which the gel is maintained can accelerate syneresis; The temperature can be low or high, according to the type of gel and its tendency to syneresis, for example, when refrigerating butter, it shows syneresis by means of liquid drops deposited on its surface; e) the pressure exerted on a gel contributes to its syneresis; d) the nature of the dispersed phase has an effect on syneresis, for example, decreasing the concentration of starch in a gel increases its syneresis; The opposite occurs in gels formed with silicic acid as the dispersed phase. [Montejano Gaitán, J. G. (2012). Estado de dispersión. En S. Baudi Dergal (Ed.), Química de los alimentos (pp. 560-561). Nahucalpan de Juárez, Edo. De México, México: Person].

On the other hand, there is a physical phenomenon called imbibition; When in contact with water, many solid substances, whether of protein origin (gelatin, albumin) or polysaccharides (pectin, soluble starches), begin to absorb it by increasing their volume, in other words, they present imbibition, the rate of which generally It decreases with the contact time between a gel and water. The main factors that affect the imbibition of a gel are pH and temperature. For example, protein gels soak more easily in slightly acidic conditions; For their part, gelatin or agar gels, when in contact with water at temperatures greater than 35° C., will present such a degree of imbibition that after a significant increase in their volume they will become thermoreversible and show liquefaction. [Montejano Gaitán, J. G. (2012). Estado de dispersión. En S. Baudi Dergal (Ed.), Química de los alimentos (pp. 560-561). Nahucalpan de Juárez, Edo. De Mexico, Mexico: Person].

From the above, it is clear that there is a growing interest in using natural products in the treatment of various diseases and that finding the various active compounds or substances that allow a desired biological effect represents an important technological challenge, likewise, their form of presentation or delivery. It is the subject of various investigations that allow obtaining the best conditions to carry out the delivery of said active compounds.

Among the commercial products that are on the market to treat IBS symptoms are:
Intestinal relaxants: meverberine, trimebutine. Libertrim®
Anticholinergics: butylhyoscine, hyoscine. Buscapina®
Calcium channel blockers: pinaverium bromide, pirenzepine, peppermint oil. Espaven®
Baking soda. Sal de uvas Picot®
Milk of magnesia. Melox®

Since there is no single medication to treat all IBS patients, 2 types of strategies have been recommended: direct treatment to the most bothersome symptom for the patient (pain, bloating, constipation, diarrhea), or to the pathophysiological mechanisms involved in the genesis of symptoms such as visceral hypersensitivity, motor alterations, dysbiosis, intestinal bacterial overgrowth, fluid homeostasis and neuroplasticity.

Limited access to some functional foods and/or nutraceuticals is currently a reality, whether due to lifestyle, availability and misinformation, the replacement of some foods that worsen IBS symptoms with others that suggest consuming make the population seek the support of nutrition professionals and currently there is no food supplement on the market that serves as support to increase the total dietary intake of bioactive compounds related to the improvement of symptoms based on the idea that In food supplements we will find a higher concentration of these compounds.

It has been reported in the scientific literature that moderate pineapple consumption does not negatively affect people's health. Likewise, it has been reported that the consumption of guava leaf extracts and walnut leaf and bark extracts are not harmful to human health; Benefits have even been reported at doses of 500 mg/kg and 300 mg/kg, respectively. Objective demonstrations of the relationship between diet and the development of the most prevalent diseases worldwide, derive from scientific knowledge of the metabolic and physiological effects of the wide diversity of natural elements; This has allowed us to promote the application and usefulness of nutraceuticals in the treatment and prevention of certain pathologies within the area of Nutrition.

It is for the reasons stated above that the purpose of the present invention is to develop a nutraceutical gel based on natural extracts, with a digestive and anti-inflammatory effect for the treatment of patients with Irritable Bowel Syndrome, which proposes an alternative food supplement for those people who suffer from it.

### BRIEF DESCRIPTION OF THE FIGURES

FIG. 1. Shows photographs of the nutraceutical composition obtained.

FIG. 2. General graph of the TPA texture profile analysis of the nutraceutical composition obtained.

### DETAILED DESCRIPTION OF THE INVENTION

The present invention relates to a nutraceutical composition, particularly in the form of a nutraceutical gel based on pineapple (Ananas comosus) and plant extracts, specifically guava (Psidium guajava) and walnut (Juglans regia) and its use for the treatment of irritable bowel. Wherein the nutraceutical composition, in the form of a nutraceutical gel, is comprised of:
a) 6.58% to 8.58% w/w citrus pectin,
b) 33.33% w/w of pineapple powder,
c) 3.33% w/w of bromelain 80 GDU,
d) 1% w/w aqueous extract of Juglans regia leaf,
e) 1% w/w aqueous extract of Psidium guajava leaf,
f) A volume of purified water until 100% w/w of the composition is complete,
g) 2.5 g of citric acid and 2 g of sodium benzoate per kilogram of mixture of the final composition.

The nutraceutical composition was tested in a pilot clinical trial where the intestinal symptomatological improvement of patients with irritable bowel syndrome was demonstrated with the consumption of the nutraceutical gel, which is why its use is proposed as an agent for the treatment of irritable bowel syndrome, demonstrating a digestive and anti-inflammatory effect.

### EXAMPLE 1

### Preparation of Nutraceutical Gel and its use for the Treatment of Patients with Irritable Bowel Syndrome

In order to illustrate the invention, without limiting it, in the following example, 1200 g of nutraceutical gel are prepared under the following process and quantities:
1. Dissolve 91 g of High Methoxyl (HM) citrus pectin in 645 mL of purified water at a temperature of 80° C. by constant stirring, adding it slowly until the citrus pectin is dissolved.
2. Let the solution from stage 1 cool until reaching a temperature of 50° C.,
3. Dissolve 400 g of pineapple powder in the mixture of stage 2 by constant stirring,
4. Dissolve 40 g of Bromelain 80 GDU in the mixture of stage 3 by constant stirring,
5. Dissolve 12 g of aqueous extract of Juglans regia leaf by constant stirring, previously obtained, by means of an aqueous extraction with the following characteristics: aceration at room temperature of 6 g of Juglans regia leaf in 300 ml of distilled water, for 24 hours later vacuum filtered and dried in an oven at 40° C. for 24 hours.
6. Dissolve 12 g of aqueous extract of Psidium guajava leaf by constant stirring, previously obtained, through an aqueous extraction with the following characteristics: Maceration at room temperature of 6 g of Psidium guajava leaf in 300 ml of distilled water, for 24 hours later vacuum filtered and dried in an oven at 40° C. for 24 hours.
7. Add 2.5 g citric acid and 2 g of sodium benzoate per kilogram of mixture final,
8. Pack in trilaminated paper envelopes.

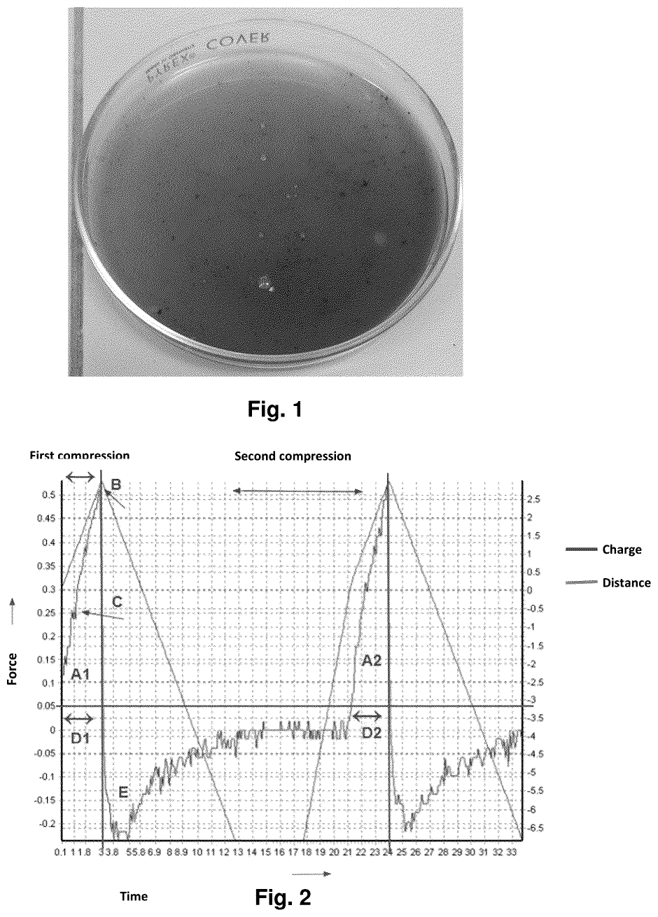

All ingredients, except for the aqueous extracts of Juglans regia leaf and Psidium guajava leaf, were obtained from Herbal and Chemical Supply. The constant stirring will be carried out using a conventional, industrial blender (for example, TAPISA® blender, 3450 rpm), mixer or any means that allows the dissolution of the pectin in the medium or the compounds and the speed varies depending on the amount. of the total nutraceutical gel to be prepared. The resulting nutraceutical gel is shown in FIG. 1.

### Bromatological Analysis of the Nutraceutical Composition

The bromatological analysis of the nutraceutical composition obtained was carried out to know the nutritional chemical composition by applying methods of the Association of Official Chemical Analysts (AOAC international) and official Mexican standards corresponding to:

### Protein Content

The protein content was determined using the Kjeldahl method (AOAC-7.033-7.037, 1984), which is based on the determination of nitrogen. This method is based on the

digestion of the product with concentrated sulfuric acid which transforms the organic nitrogen into ammonium ions, in the presence of copper sulfate as a catalyst, addition of an alkali, distillation of the released ammonia into an excess of sulfuric acid solution and subsequent titration of the excess acid with sodium hydroxide solution. Since it is a method for determining total Nitrogen, through which % of crude protein (total) can be obtained from an organic sample. The percentage of nitrogen obtained can be transformed into a % of crude protein, thanks to the conversion factor of 6.25 which comes from the consideration that most proteins have an approximate amount of 16% nitrogen. That is, in 100 grams of sample, 16% correspond to nitrogen.

### Total Fat Content

On the other hand, the Soxhlet method (AOAC 1990-920.39C) was used to obtain the total fat content of the sample, since based on a property of the substance that we are interested, in this case fat, allows us to indirectly quantify its presence in said product. This analysis is also known as gravimetric. In this way, the method records the weight of the food sample at two key moments: at the beginning, when the food still contains the substance of interest; and at the end, when it has lost part of its composition. Thus, by weight difference, it is possible to estimate the percentage of the compound that was analyzed.

### Trans Fat Content

Likewise, the method used for trans fats was gas chromatography, and according to the Official Association for Chemical Analysis (A.O.A.C 925.10), it is one of the most appropriate and accepted by the scientific community for the quantification of TFA in food, is widely used due to the exceptional resolution characteristics of the method and the wide variety of detectors that can be used. Sample preparation generally includes isolation of solutes from foods by distillation, preparative chromatography (including solid phase extraction), or liquid extraction. Some solutes can be analyzed directly. The CG consists of a gas source and regulators (flow and pressure control), injection port, column, oven, detectors, and data processing and storage system. It is necessary to optimize the gas flow and temperature profile during separation.

### Dietary Fiber Content

In addition, the enzymatic method was used to determine dietary fiber (AOAC 985.29, 2000) and is mainly based on digesting proteins and carbohydrates with enzymes, the remainder is allocated to Dietary Fiber after discounting the ash content. and remaining proteins. Dietary Fiber may be determined alone, or, by alcohol precipitation, SDF may be included and determined separately or together. The main advantages of these methods are that they are relatively accurate and precise compared to other procedures. However, they do not give detailed information about the components of Total Dietary Fiber. These methods are considered the most suitable for routine analysis for fiber labeling and quality control purposes.

### Sodium Content

The method implemented to determine the sodium of the product was through the flame photometry technique and this is mainly a quantitative analysis method, it is one of the simplest and most accurate to analyze sodium, it is based on the excitation suffered by the electrons of an atom when a source of thermal energy from the combustion of a flame is applied to them; Upon returning to the ground state, atoms emit a certain amount of energy in a quantified form. Specifically, in flame atomic emission spectrometry only lines are observed, well defined for each atom, caused by transitions between the lowest energy levels and the ground state.

### Total Carbohydrate Content

The total carbohydrate content has been determined by difference data using the Weende proximal system of analysis. The system consists of the analytical determination of water (moisture), ash, crude fat (ether extraction), crude protein and crude fiber. Nitrogen-free extract (NFL), which roughly represents sugars and starches, is calculated by the difference rather than measured by analysis. And more specific for sugars present in the sample using the chromatography method, chromatography is a physical separation method, in which the components to be separated are distributed between two phases, one stationary (stationary phase) and another that moves (phase). mobile) in a defined direction.

All the results obtained from the nutraceutical composition made are shown in table 2.

TABLE 2
National Nutrient Table (NOM-051) of the nutraceutical
composition obtained in the present invention.

| Parameter | Methodology | Resultado | |
|---|---|---|---|
| Portion | | 100 | g |
| Total Fats | Soxhtel | 0.68 | g |
| Saturated fats | Chromatography | 0.23 | g |
| Trans Fat | Chromatography | 0 | mg |
| Polyunsaturated Fat | Chromatography | N.A. | |
| Monounsaturated Fat | Chromatography | N.A. | |
| Cholesterol | Chromatography | N.A | |
| Sodium | Photometry | 49.22 | mg |
| Total carbohydrates | Difference | 39.8 | g |
| Dietary fiber | Enzymatic | 16.35 | g |
| Sugars | Chromatography | 11.57 | |
| Added Sugars | Chromatography | 11.57 | |
| Protein | Kjeldhal | 1.18 | g |

From the results obtained in the bromatological analysis of the nutraceutical composition, the nutritional declaration of the composition was made and is shown in table 3.

TABLE 3
Nutritional declaration of the nutraceutical
composition obtained in the present invention.
Nutritional Declaration

| Energy content (445 kJ) | 100 g: 105 Kcal | | 15 g: 24 Kcal (890 kJ) | |
|---|---|---|---|---|
| Proteins | 1 | g | 0 | g |
| Total fats | 1 | g | 0 | g |
| Saturated fats | 0 | g | 0 | g |
| Trans fats | 0 | mg | 0 | mg |
| Available carbohydrates | 23 | g | 3 | g |
| Sugar | 12 | g | 2 | g |
| Added Sugar | 0 | g | 0 | g |
| Dietary fiber | 16 | g | 2 | g |
| Sodium | 50 | mg | 8 | mg |

### Microbiological Analysis

A microbiological analysis type study was carried out on the nutraceutical composition obtained in the present invention in which a comparison was made between the laboratory results obtained and the microbiological criteria established within the aforementioned Standards, which determined that the microorganisms Escherichi coli, Staphylococcus aureus, Coliform bacteria count, Molds and Yeast count, which allowed us to observe in Table 4 that the parameters are within the permitted limits.

TABLE 4
Microbiological analysis of the nutraceutical
composition obtained in the present invention.

| Parameter | Methodology | Result |
|---|---|---|
| Aerobic Mesophilic | NOM-092-SSA1-1994 | 140 UC/g “Estimated |
| Bacteria Count | | value” |
| Coliform Bacteria Count | NOM-092-SSA1-1994 | <10 UFC/g |
| Mold Account | NOM-092-SSA1-1994 | <10 UFC/g |
| Yeast Count | NOM-092-SSA1-1994 | <10 UFC/g |
| NMP Total Coliforms | NOM-092-SSA1-1994 | N.A |
| NMP Fecal Coliforms | NOM-092-SSA1-1994 | N.A |
| Salmonella | NOM-092-SSA1-1994 | Absence 25 g |
| Escherichia coli | NOM-092-SSA1-1994 | <3 UFC/g |
| Staphylococcus aureus | NOM-092-SSA1-1994 | <10 UFC/g |
| Listeria spp | NOM-092-SSA1-1994 | N.A |
| Vibro cholerae | NOM-092-SSA1-1994 | N.A |
| Sporulated Mesophilic | NMX-F-403-S-1981 | N.A |
| Count | | |

### Physicochemical Analysis

Physicochemical analyzes were carried out on the nutraceutical composition obtained in the present invention, which made it possible to verify that the values of total hardness (NMX-F-517-1992), hydrogen potential (NMX-AA-008-SCFI-2016) and chloride (NMX-AA-073-SCFI-2001) are within the permitted limits according to their respective standards. The results together with the parameters and methodology used are described in table 5.

TABLE 5
Physicochemical analysis of the nutraceutical
composition obtained in the present invention.

| | | | Maximum |
|---|---|---|---|
| Parameter | Methodology | Result | value |
| Conductivity | NMX-AA-093- | N.A. | — |
| | SCFI-200 | | |
| Total or permanent | NMX-F-517-1992 | N.A. | 500 p.pm de |
| hardness | | | CaCO₂ |
| Calcium hardness | NMX-F-517-1992 | N.A. | — |
| Magnesium | NMX-AA-036- | N.A. | — |
| hardness | SCFI-2001 | | |
| Alkalinity F | NMX-AA-036- | N.A. | ** |
| | SCFI-2001 | | |
| Alkalinity M | NMX-AA-073- | N.A. | 250 ppm of Cl |
| | SCFI-2001 | | |
| Chlorides | NMX-AA-008- | N.A. | 6.5-8.5 unidades |
| | SCFI-2016 | | de pH |
| Hydrogen Potential | Potentiometry | 4.18 pH | |
| (pH) | | Unities | |
| Total dissolved | NMX-AA-034- | N.A. | 1,000 ppm |
| solids | SCFI-2015 | | |
| Acidity | Volumetry | | — |
| Salt | Volumetry | N.A. | — |
| Density | Gravimetry | N.A. | — |
| Peroxide Index | Volumetry | N.A. | — |
| Residual Free | Volumetry | N.A. | 0.8-1.5 ppm Cl₂ |
| Chlorine | | | |
| Determiantion of | NMX-AA-084- | N.A. | ≥99.4% |
| total sugars | SCFI-2004 | | |
| Water activity | AOAC 978.18 | N.A. | — |

Observations & symbology
*** Total Alkalinity = 300 ppm (sum of Alkalinity F + Alkalinity M) ppm = parts per million CaCO₂ = calcium carbonate Cl = chlorides
NOM-127-SSA1-1994, Environmental health. Water for human use and consumption.
Permissible limits of quality and treatment to which the water must be subjected for its purification.
□Methods accredited by EMA for this laboratory
NA Not applicable or not determined

### Rheological Analysis

A 2-cycle texture profile analysis was performed in triplicate with 1% loading sensitization to the nutraceutical composition obtained in the present invention by means of a Brookfield CT3 Texturometer, the results are shown in FIG. 2. The cylindrical samples of The gels were subjected to two puncture cycles with an acrylic cylinder of 1.27 cm in diameter and with a penetration distance of 43% with respect to the original height of the gel, at a speed of 1.0 mm/s with a waiting time between 5 second cycles. The contact force with the sample was 0.05 N. The composition presented the results in table 6.

TABLE 6
Texture Profile
Cycle 1

| Hardness | 0.61N | |
|---|---|---|
| Deformation | 2.93 | mm |
| Adhesiveness | 1.43 | mJ |
| Extensibility | 1.20 | mm |

| | |
|---|---|
| Fractureability | 0.23N |

Cycle 2

| Hardness | 0.61N | |
|---|---|---|
| Cohesiveness | 0.68 | |
| Elasticity | 2.29 | mm |

| | |
|---|---|
| Gummyness | 0.41N |
| Chewyness | 0.94 |
| Chewyness | 0.94 |

### Pilot Clinical Trial

A pilot clinical trial was carried out with the nutraceutical composition obtained in the present invention, complying with ethical and scientific aspects in accordance with the Regulations of the General Health Law on Health Research, classifying the present research according to article 17; section II as an investigation with minimal risk. As well as the requirements established by the biosafety department of the UANL Medical Services Clinic. Each patient signed an informed consent form in which they were informed: the purpose of the study, its duration, inclusion criteria as a study subject, what their participation consisted of and the procedures to be performed, possible risks or discomforts, what to do. in case you feel any type of discomfort, possible benefits for the patient and for the development of the present invention, the exclusive use of your samples and data collected for the trial, your rights as a study subject, the absolute confidentiality of the data collected and results of the analysis of the samples, as well as the relevant contacts if there is any type of doubt in the event of agreeing to participate.

The evaluation of the nutraceutical composition obtained in the present invention, in the form of nutraceutical gel, was carried out through a randomized and controlled clinical trial with patients diagnosed with irritable bowel syndrome, considering intentional non-probabilistic sampling.

The trial was carried out with 2 groups: Group 1 (8 patients): administration of the nutraceutical gel object of the present invention; This group was given one sachet of the nutraceutical gel (15 g) daily for 14 days, in the mornings; and Group 2 (8 patients):
administration of a placebo gel (only with pectin, pineapple flavoring and water); This group was given one sachet (15 g) daily for 14 days, in the mornings.

At the end of the test time, the digestive effect of the nutraceutical composition was measured through the impact on the symptoms by the Bristol stool scale or Bristol stool chart, which is a visual table intended to classify the shape of human stools in seven groups which was developed by Ken W. Heaton and Stephen J. Lewis at the University of Bristol, where type 1 refers to separate hard pieces, which are passed with difficulty (significant constipation), type 2 which refers to stools like a sausage made up of fragments (mild constipation), type 3 to black pudding-shaped stools with cracks on the surface (normal), type 4 to sausage-or snake-shaped stools, smooth and white (normal), type 5 to pieces pasty mass with defined edges (lack of fiber), type 6 to pasty fragments, with irregular edges (slight diarrhea) and type 7 to watery stools, without solid pieces, completely liquid (significant diarrhea). [Lewis S J, Heaton K W (1997). «Stool form scale as a useful guide to intestinal transit time». Scandinavian Journal of Gastroenterology (en inglés) 32 (9): 920-4. PMID 9299672]. An improvement in normal stools was obtained in 100% of Group 1 and 87.5% of Group 2.

The effect of the nutraceutical composition on the state of gravity of the syndrome was also evaluated through an IBS severity questionnaire for each group before and after the intervention, the results are shown in tables 7, 8 and 9, and measure the abdominal pain, abdominal distension and bowel habit in the two study groups (Almansa, Cristina, García-Sánchez, Raquel, Barceló, Marta, Díaz-Rubio, Manuel, & Rey, Enrique. (2011). Traducción, adaptación cultural y validación al español del cuestionario de gravedad del síndrome de intestino irritable (Irritable Bowel Syndrome Severity Score). Revista Española de Enfermedades Digestivas, 103 (12), 612-618. https://dx.doi.org/10.4321/S1130-01082011001200002).

TABLE 7
Abdominal pain (tummy pain) before and
after administration of the gels.

| Groups | |
|---|---|
| Group 1 | Group 2 |

| | Antes | Después | Antes | Después |
|---|---|---|---|---|
| Without pain | 1 | 7 | 2 | 2 |
| Not serious | 5 | 1 | 3 | 4 |
| Something Serious | 2 | 0 | 3 | 2 |
| Serious | 0 | 0 | 0 | 0 |
| Very serious | 0 | 0 | 0 | 0 |
| Total | 8 | 8 | 8 | 8 |

TABLE 8
Abdominal distension (swollen, inflated or
tense belly) before and after intervention.

| Groups | |
|---|---|
| Group 1 | Group 2 |

| | Before | After | Before | After |
|---|---|---|---|---|
| Si* | 6 | 1 | 5 | 2 |
| No | 2 | 7 | 3 | 5 |
| Total | 8 | 8 | 8 | 8 |

*100% of the participants in both groups reported that the abdominal distensions were Not Serious.

TABLE 9
Bowel habit (go potty) measure of bowel habit
satisfaction before and after the intervention.

| Groups | |
|---|---|
| Group 1 | Group 2 |

| | Before | After | Before | After |
|---|---|---|---|---|
| Very unsatisfied | 0 | 0 | 0 | 0 |
| Unsatisfied | 8 | 0 | 8 | 1 |
| Satisfied | 0 | 8 | 0 | 7 |
| Very satisfied | 0 | 0 | 0 | 0 |
| Total | 8 | 8 | 8 | 8 |

Based on the results of the pilot clinical study developed to demonstrate the intestinal symptomatological improvement of patients with irritable bowel syndrome with the consumption of the nutraceutical gel, a decrease in general symptoms can be reflected thanks to the use of the nutraceutical gel comparing group 1 with group 2 of the present clinical trial, showing a digestive and/or anti-inflammatory effect.

### Pilot Sensory Analysis of the Nutraceutical Gel

A survey was applied to the patients who consumed the nutraceutical gel, obtaining the following results shown in table 10.

TABLE 10
Pilot sensory analysis of the nutraceutical gel
TASTE: VERY GOOD

| | 1 | 2 | 3 | 4 | 5 | |
|---|---|---|---|---|---|---|
| Very bad | 0% | 0% | 0% | 72% | 28% | Excellent |

TEXTURE: EXCELLENT

| | 1 | 2 | 3 | 4 | 5 | |
|---|---|---|---|---|---|---|
| Very bad | 0% | 0% | 0% | 14% | 85% | Excellent |

COLOR: BUENO

| | 1 | 2 | 3 | 4 | 5 | |
|---|---|---|---|---|---|---|
| Very bad | 0% | 14% | 44% | 28% | 14% | Excellent |

SMELL: EXCELLENT

| | 1 | 2 | 3 | 4 | 5 | |
|---|---|---|---|---|---|---|
| Very bad | 0% | 0% | 0% | 28% | 44% | Excelent |

OVERALL OPINION: EXCELLENT

| | 1 | 2 | 3 | 4 | 5 | |
|---|---|---|---|---|---|---|
| Very bad | 0% | 0% | 0% | 42% | 58% | Excellent |

1. A nutraceutical composition based on pineapple and plant extracts characterized by comprising:
a) 6.58% to 8.58% w/w of citrus pectin,
b) 33.33% w/w of pineapple powder,
c) 3.33% w/w of bromelain 80 GDU,
d) 1% w/w of aqueous extract of Juglans regia leaf,
e) 1% w/w of aqueous extract of Psidium guajava leaf,
f) A volume of purified water to complete 100% w/w of the composition.

2. The nutraceutical composition based on pineapple and plant extracts according to claim 1 characterized by the addition of 2.5 g of citric acid and 2 g of sodium benzoate per kilogram of the nutraceutical composition mixture.

3. The nutraceutical composition based on pineapple and plant extracts according to claim 1 characterized by the citrus pectin being High Methoxyl (HM).

4. The nutraceutical composition based on pineapple and plant extracts according to claim 1 characterized by having a gel form.

5. The nutraceutical composition based on pineapple and plant extracts described in claims 1 to 4 for use as an anti-inflammatory agent for the treatment of irritable bowel syndrome.
